(19) United States
(12) Patent Application Publication
(10) Pub. No.: US 2021/0267248 A1
(43) Pub. Date: Sep. 2, 2021

(54) Nutritional Compositions for Treating Cancer and Side Effects of Cancer Conventional Treatments

(71) Applicants: David Avidar, Hod Hasharon (IL); Yair Mievski, Jerusalem (IL); Avraham Mivski, Beitar Elit (IL)

(72) Inventors: David Avidar, Hod Hasharon (IL); Yair Mievski, Jerusalem (IL); Avraham Mivski, Beitar Elit (IL)

(21) Appl. No.: 17/180,804

(22) Filed: Feb. 21, 2021

(30) Foreign Application Priority Data
Feb. 27, 2020 (IL) 272974

(51) Int. Cl. A23L 33/15; A61P 35/00; A23L 29/00

(52) U.S. Cl. CPC A23L 33/15; A23L 29/015; A61P 35/00

(57) ABSTRACT

A nutritional composition for the treatment of cancer and side effects of conventional cancer treatment that includes gold powder, magnesium, sodium, calcium and vitamin B6.

### TECHNICAL FIELD

The present invention relates to nutritional compositions for the treatment of cancer and side effects of conventional cancer treatments, and the method involved. These compositions comprise Gold Powder, Magnesium, Sodium, Calcium and Vitamin B6.

### BACKGROUND ART

It is known that there is a real need for an effective cancer treatment as well as for treatment of the side effects of the conventional cancer treatment. The present invention discloses a method and a composition for treatment of cancer and the side effects of cancer treatment.

### DESCRIPTION OF THE DRAWINGS

The intention of the drawings attached to the application is not to limit the scope of the invention and its application. The drawings are intended only to illustrate the invention and they constitute only one of its many possible implementations.

FIG. 1 schematically illustrates the capsule (100) with gold powder, magnesium, sodium, calcium and vitamin B6.

### THE INVENTION

The present invention is based on the use of a novel combination of materials in the treatment of cancer, and therefore the main object of the present invention is to provide a nutritional composition for the treatment of cancer that includes gold powder, magnesium, sodium, calcium and vitamin B6. The gold powder, the magnesium, the sodium, the calcium and the vitamin B6 can be purchased in the market as a powder for example and it is possible to mix them together before administration orally to the patient.

The term “treatment” is used conventionally and refers to the purpose to diminish, retard, or stop the growth, metastasis, or spread of cancer cells.

The nutritional composition is highly effective when the powder gold is in the amount of about 7% by weight of the composition, the magnesium is in the amount of about 65% by weight of the composition, the sodium is in the amount of about 13% by weight of the composition, the calcium is in the amount of about 13% by weight of the composition, and the vitamin B6 is in the amount of about 2% by weight of the composition.

It is preferably to administer the nutritional composition when it is in a capsule which should be placed under the tongue until the capsule and the composition dissolves. Therefore the present invention in particularly refers to composition that is encapsulated within a capsule and suitable for oral administration and wherein the powder gold is in the amount between 5 to 15 micrograms, the magnesium is in the amount between 50 to 150 micrograms, the sodium is in the amount between 10 to 30 micrograms, the calcium is in the amount between 10 to 30 micrograms, and the vitamin B6 is in the amount between 1.5 to 4.6 micrograms. However, the term “administered” in the present application and in the claims means that the composition is delivered to the subject by any means or route which is effective to achieve the desired result.

The present invention also relates to the method of treating cancer by administering to a patient in need thereof an effective amount of the nutritional composition. The effective amount is correlated to the stage of the cancer. For cancer in stage one the amount should be between 77 to 230 micrograms per day, which can be for example one capsule as stated above. For cancer in stage two the amount should be between 154 to 460 micrograms per day, which can be for example two capsules. For cancer in stage three the amount should be between 231 to 690 micrograms per day, which can be for example three capsules, and for cancer in stage four the amount should be between 308 to 920 micrograms per day, which can be for example four capsules. It is recommended that the patient will take the capsules everyday as long as he or she still has the cancer.

As mentioned above, all the ingredients of the composition can be purchased in the free market and from which the composition can be prepared. It is also possible to purchase the capsules in the market and it is preferably to use capsules that dissolve under the tongue. Then, it is possible to fill the capsule with the composition in the amounts as explained above.

Many cancer patients received the composition and after a few months some recovered completely and the health of the rest improved significantly. Here below some cases that illustrates the effectiveness of the composition.

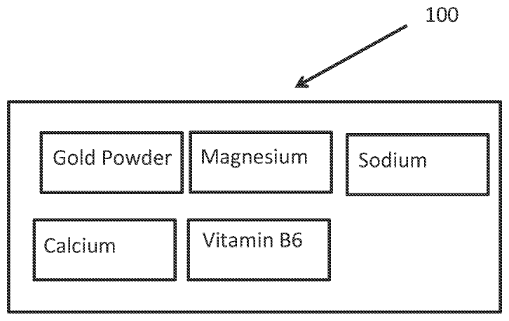

FIG. 1 schematically illustrates the capsule (100) with gold powder, magnesium, sodium, calcium and vitamin B6.

Example number 1: a middle-aged man was terminally ill with cancer and the doctors predicted he would die within a few months. The patient took one capsule every day for six months and he is fully recovered.

Example number 2: A five-years-old child with cancer was confined to the bed without the possibility to be active physically. The child took a capsule every day, and after two months he got better and has started to play and ran like a healthy child, and after another two months he has fully recovered.

Example number 3: A 62-year-old woman that had a liver cancer with metastasis. Her blood test indicated ca19.9 at a degree of 9300. She was confined to the bed, with eating difficulties (lack of appetite), and with difficulties to talk due to body fatigue. She started taking 4 capsules a day and after one month the ca19.9 degree was 3,300, and after six months from the start of the treatment, she were active, energetic, began to eat reasonable meals, functioned at home chores, shopping on her own and in a recovery stage.

Example number 4: A 55-year-old man with head and lung cancer that was confined to bed, unable to speak, and doctors estimated that he would die within a month. The patient took 4 capsules per day and after four months his health has improved and after two more months the cancer is completely gone.

Example number 5: A sixty-year-old woman with lung cancer, that was connected most of the time to an oxygen mask and she couldn't lie down in the light of a sultry cough. Chemotherapy did not improve her condition. A month after she began taking the capsules (4 per day) she began to lie down in bed, no longer needed oxygen masks, and her medical condition improved.

The object of the present invention is also to provide a method for the treatment of side effects of conventional cancer treatment and the side effects of the cancer itself, by administering to the patient in need a gold powder. The term “treatment” is also refers to the purpose of reducing the side effects of the conventional cancer treatment and of the side effects of the cancer itself, such as weakness, physical dysfunction, loss of appetite and nausea.

It is preferably to administer the gold powder when it is within a capsule and suitable for oral administration and wherein the powder in each capsule is in the amount between 5 to 920 micrograms. It is recommended that the patient will take the capsules everyday as long as he or she still has the side effects of the cancer treatment or the side effects of the cancer itself.

What we claim is:

1. A nutritional composition for the treatment of cancer, comprising: gold powder, magnesium, sodium, calcium and vitamin B6.

2. The composition of claim 1, wherein the powder gold is in the amount of about 7% by weight of the composition, the magnesium is in the amount of about 65% by weight of the composition, the sodium is in the amount of about 13% by weight of the composition, the calcium is in the amount of about 13% by weight of the composition, and the vitamin B6 is in the amount of about 2% by weight of the composition.

3. The composition of claim 1, which is encapsulated within a capsule and suitable for oral administration wherein the powder gold is in the amount between 5 to 15 micrograms, the magnesium is in the amount between 50 to 150 micrograms, the sodium is in the amount between 10 to 30 micrograms, the calcium is in the amount between 10 to 30 micrograms, and the vitamin B6 is in the amount between 1.5 to 4.6 micrograms.

4. A method of treating cancer, comprising administering to a patient in need thereof an effective amount of a nutritional composition according to claim 1, wherein said amount is between 77 to 230 micrograms per day when the cancer is at stage one, between 154 to 460 micrograms per day when the cancer is at stage two, between 231 to 690 micrograms per day when the cancer is at stage three, and between 308 to 920 micrograms per day when the cancer is at stage four.

5. The method of treating cancer, comprising administering to a patient in need thereof one capsule according to claim 3 every day when the cancer is at stage one, two capsules when the cancer is at stage two, three capsules when the cancer is at stage three and four capsules when the cancer is at stage four.
